(19) United States
(12) Patent Application Publication
(10) Pub. No.: US 2010/0114171 A1
(43) Pub. Date: May 6, 2010

(54) MULTI-PLANAR SPINAL FIXATION ASSEMBLY WITH LOCKING ELEMENT

(75) Inventors: Oheneba BOACHIE-ADJEI, Briarcliff, NY (US); Michael BARRUS, Ashburn, VA (US); Larry McCLINTOCK, Gore, VA (US)

Correspondence Address: CARTER, DELUCA, FARRELL & SCHMIDT, LLP, 445 BROAD HOLLOW ROAD, SUITE 420, MELVILLE, NY, 11747, US

(73) Assignee: K2M, INC., Leesburg, VA (US)

(21) Appl. No.: 12/612,762

(22) Filed: Nov. 5, 2009

Related U.S. Application Data
(60) Provisional application No. 61/198,374, filed on Nov. 5, 2008.

(51) Int. Cl. A61B 17/70; A61B 17/86

(52) U.S. Cl. 606/264; 606/308

(57) ABSTRACT

A spinal fixation assembly includes a spinal rod, a first spinal fixation device, and a locking element. The spinal rod defines a longitudinal axis. The first spinal fixation device includes a coupling, a screw, and a collet. The coupling has an opening extending therethrough. The screw is mounted to the coupling and positionable within a first vertebral body. The collet is receivable in the opening of the coupling and defines a saddle for engaging the spinal rod. The locking element is mounted to the spinal rod and is configured to prevent axial translation of the spinal rod in one or both directions along the longitudinal axis thereof.

### CROSS REFERENCE TO RELATED APPLICATIONS

This application claims priority to, and the benefit of, U.S. Provisional Patent Application Ser. No. 61/198,374, filed Nov. 5, 2008, the contents of which are hereby incorporated by reference in their entirety.

### BACKGROUND

1. Technical Field

The present disclosure relates generally to orthopedic surgery and in particular to devices and prosthesis for stabilizing and fixing the bones and joints of the body.

2. Background of Related Art

It is a common surgical procedure to stabilize and fix bones and bone fragments in a particular spatial relationship with fixation devices to correct the location of skeletal components due to injury or disease. This can be accomplished by using a number of fixation devices such as bone pins, anchors, or screws placed in bone across a discontinuity (e.g., a fracture) in the bone, bone fragments, adjacent vertebrae, or joints. These fixation devices can be connected by a rod to maintain a desired spatial relationship. In some cases, these fixation devices may be permanently implanted. In other cases, these fixation devices may be implanted only as a temporary means of stabilizing or fixing the bones or bone fragments. It is also common that fixation devices that are intended to be permanently implanted require subsequent modifications as the dynamics of a patient's condition warrant.

Spinal fixation devices are widely employed in surgical processes for correcting spinal injuries and diseases. These devices commonly employ longitudinal link rods (e.g., spinal rods) secured to vertebrae by spinal bone fixation fasteners such as pedicle screws, hooks and others.

On occasion, the rod may dislocate from the spinal fixation device under bodily forces experienced after implantation. Such dislocation can be caused either by axial slip, i.e., sliding of the rod end through the spinal fixation device along the axis of the rod, or radial displacement of the rod out of the screw. Either type of dislocation can happen with any type of spinal fixation device, including both taper lock style screws and set screw style screws.

### SUMMARY

The present disclosure relates to a spinal fixation assembly including a spinal rod, one or more spinal fixation devices, and a locking element. The one or more spinal fixation devices include a coupling, a screw, and a collet. The coupling has an opening extending therethrough. The screw is mounted to the coupling and positionable within a first vertebral body. The collet is receivable in the opening of the coupling and defines a saddle for engaging the spinal rod.

The spinal rod defines a longitudinal axis. The spinal rod is positioned transverse to the screw upon engagement with the saddle of the collet.

The locking element is mounted to the spinal rod and is configured to prevent axial translation of the spinal rod in one or both directions along the longitudinal axis thereof. The locking element includes a body defining a passage adapted to receive the spinal rod therethrough in locking engagement therewith. The locking element includes a locking screw for selectively locking the locking element to the spinal rod. The locking screw rotates into locking engagement with the spinal rod. The locking screw is disposed in threaded engagement with a threaded bore defined within the body of the locking element. The width of the locking element is greater than the width of the saddle such that when the locking element abuts the saddle, the locking element prevents the spinal rod from axially translating in one or both directions.

The spinal fixation assembly may include a second spinal fixation device having a collet defining a saddle wherein the spinal rod is positionable within the saddles of each spinal fixation device.

In one aspect, a spinal fixation assembly includes one or more spinal fixation devices and a spinal rod. The one or more spinal fixation devices include a coupling, a screw, and a collet. The coupling has an opening extending therethrough. The screw is mounted to the coupling and positionable within a first vertebral body. The collet is receivable in the opening of the coupling and defines a saddle. The spinal rod has one or more locking elements configured to prevent axial translation of the spinal rod in one or both axial directions when the spinal rod is positioned within the saddle of the collet of the spinal fixation device. The one or more locking elements may be an annular ring around the spinal rod. The diameter of the one or more locking elements is greater than the width of the saddle such that when the one or more locking elements abut the saddle, the one or more locking elements prevent the spinal rod from axially translating in one or both axial directions.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1A is a top perspective view of one embodiment of a spinal fixation device having a taper lock;

FIG. 1B is a front view of the spinal fixation device of FIG. 1A;

FIG. 1C is an exploded side view of the spinal fixation device of FIG. 1A with parts separated illustrating a pedicle screw, a coupling, a collet, and a pin;

FIG. 2A is a front view of the coupling;

FIG. 2B is a top perspective view of the coupling of FIG. 2A;

FIG. 3A is a front view of the collet;

FIG. 3B is a top perspective view of the collet of FIG. 3A;

FIG. 4A is a side view of the pedicle screw;

FIG. 4B is a top view of the pedicle screw of FIG. 4A;

FIG. 5 is a front perspective view of the pin;

FIG. 6 is a cross-sectional view of the spinal fixation device;

FIG. 7 is a side view of one embodiment of spinal fixation assembly including first and second spinal fixation devices, a spinal rod, and a locking element in accordance with the present disclosure;

FIG. 8A is a front view of the locking element of the spinal fixation assembly of FIG. 7, the locking element including a body and a locking screw;

FIG. 8B is an exploded front view of the locking element of FIG. 8A

FIG. 9 is a perspective view of the body of the locking element of FIG. 8A;

FIG. 10 is a perspective view of the locking screw of the locking element of FIG. 8A;

FIG. 11 is a top view of one embodiment of a spinal fixation assembly including first and second spinal fixation devices and an embodiment of a spinal rod in accordance with the present disclosure;

FIG. 12 is a side view of the spinal rod of FIG. 11;

FIG. 12A is an enlarged side view of the detailed area A of FIG. 12; and

FIG. 13 is a perspective view of another embodiment of a spinal fixation assembly in accordance with the present disclosure.

### DETAILED DESCRIPTION OF THE EMBODIMENTS

Various embodiments of the presently disclosed spinal fixation assembly will now be described in detail with reference to the drawings, wherein like reference numerals identify similar or identical elements. In the drawings and in the description that follows, the term “proximal,” will refer to the end of a device that is closest to the operator, while the term “distal” will refer to the end of the device that is farthest from the operator. In addition, the term “cephalad” is used in this application to indicate a direction toward a patient's head, whereas the term “caudad” indicates a direction toward the patient's feet. Further still, for the purposes of this application, the term “medial” indicates a direction toward the middle of the body of the patient, whilst the term “lateral” indicates a direction toward a side of the body of the patient (i.e., away from the middle of the body of the patient). The term “posterior” indicates a direction toward the patient's back, and the term “anterior” indicates a direction toward the patient's front. In the following description, well-known functions or constructions are not described in detail to avoid obscuring the present disclosure in unnecessary detail.

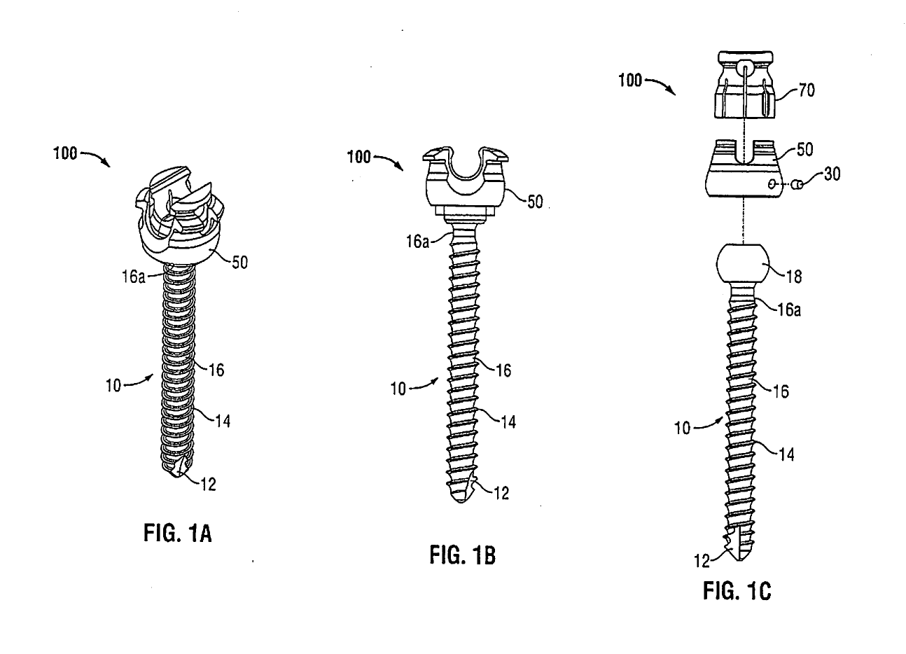

Referring initially to FIGS. 1A-1C, in which like reference numerals identify similar or identical elements, a spinal fixation device is generally designated as 100. The spinal fixation device 100 includes a pedicle screw 10, a pin 30, an outer housing or coupling 50, and an inner housing or collet 70. One example of such a spinal fixation device is disclosed in International Application Number PCT/US2008/080682, the entire contents of which are hereby incorporated by reference herein. While a taper lock screw as shown and described herein is preferred, the locking element of the present disclosure may find application with the use of other types of spinal fixation devices, such as set screw type fixation devices. One example of such as set screw type fixation device is shown in U.S. Pat. No. 5,733,286, the entire contents of which are incorporated by reference herein.

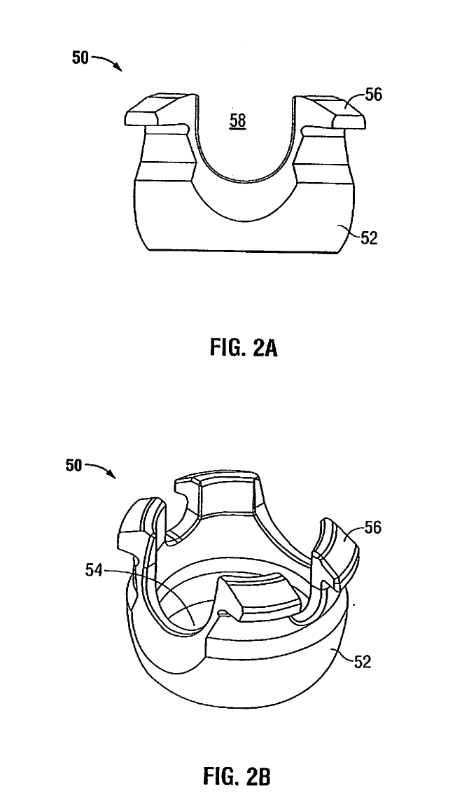

Referring now to FIGS. 2A and 2B, the coupling 50 includes an annular body portion 52 having an opening 54 extending axially therethrough. Additionally, the coupling 50 includes a plurality of fingers 56 that are located in opposing regions of the coupling 50 and define a saddle 58 having a generally U-shaped configuration. The U-shaped saddle 58 is configured and dimensioned for receiving a rod “R” (see FIG. 7).

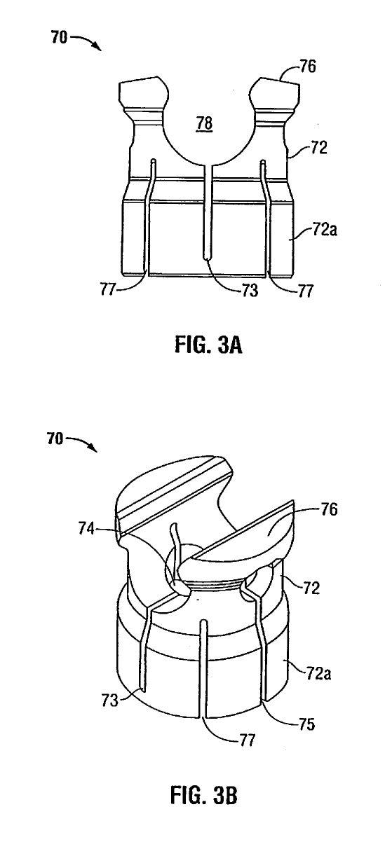

As shown in FIGS. 3A and 3B, the collet 70 has a generally cylindrical body portion 72 with an opening 74 extending axially therethrough. A pair of upstanding wings 76 defines a saddle 78 having a generally U-shaped configuration. The saddle 78 is configured and dimensioned for receiving the rod “R.” The body portion 72 includes a slot 73 that extends from the nadir of the saddle 78 towards the bottom of the body portion 72 and essentially bisects the body portion 72 along a central axis, and defines left and right sections of the body portion as viewed in FIG. 3A. Preferably, the slot 73 does not extend all the way through the body portion. Although less desirable, such a full slot could be used. This arrangement permits each of the wings 76 to flex towards and away from each other. The dimensions of the saddle 78 vary according to the flexure of the wings 76. As the wings 76 are moved closer to each other, the saddle 78 decreases in size and when the wings 76 are moved away from each other, the saddle 78 increases in size. Allowing the saddle 78 to vary in size permits the collet 70 to accommodate rods having differing outside diameters. Alternatively, compressing the wings 76 towards each other increasingly engages the outer surface of a rod “R” located in the saddle 78, thereby frictionally securing the rod “R” in a desired position.

In addition, the body portion 72 includes a plurality of grooves 75 that extend to the bottom of the body portion 72 and which are open at the bottom of the body portion 72. The grooves 75 extend vertically into each of the wings 76, and define front and rear portions of the body portion 72. As configured, the grooves 75 permit the front and rear sections of the body portion 72 to flex relative to the grooves 75 along the axis defined by the slot 73. The body portion 72 also includes a plurality of notches 77 that are open at the bottom surface of the body portion 72 and extend vertically towards the wings 76. The notches 77, in combination with the slot 73 and the grooves 75, allow arcuate sections 72a of the body portion 72 to flex inwards and outwards from an initial position in response to compressive and tensile forces applied to the sections 72a.

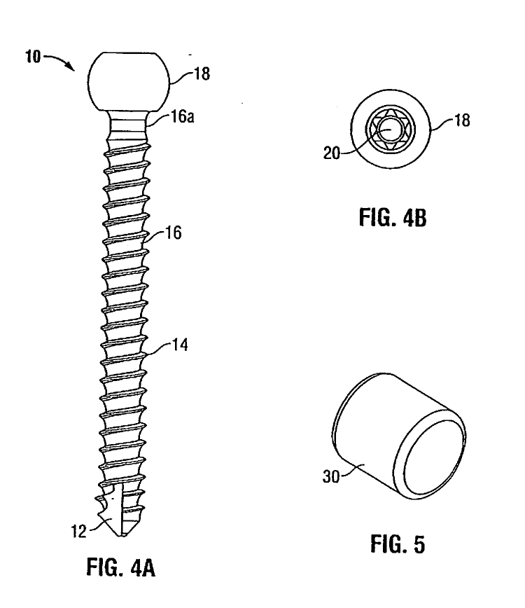

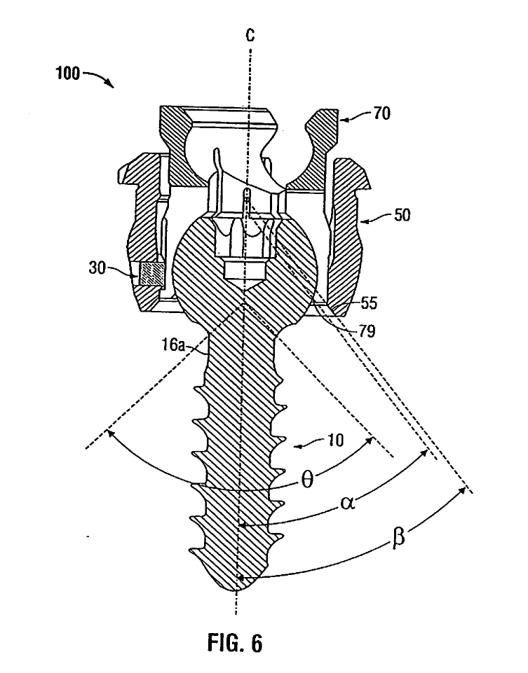

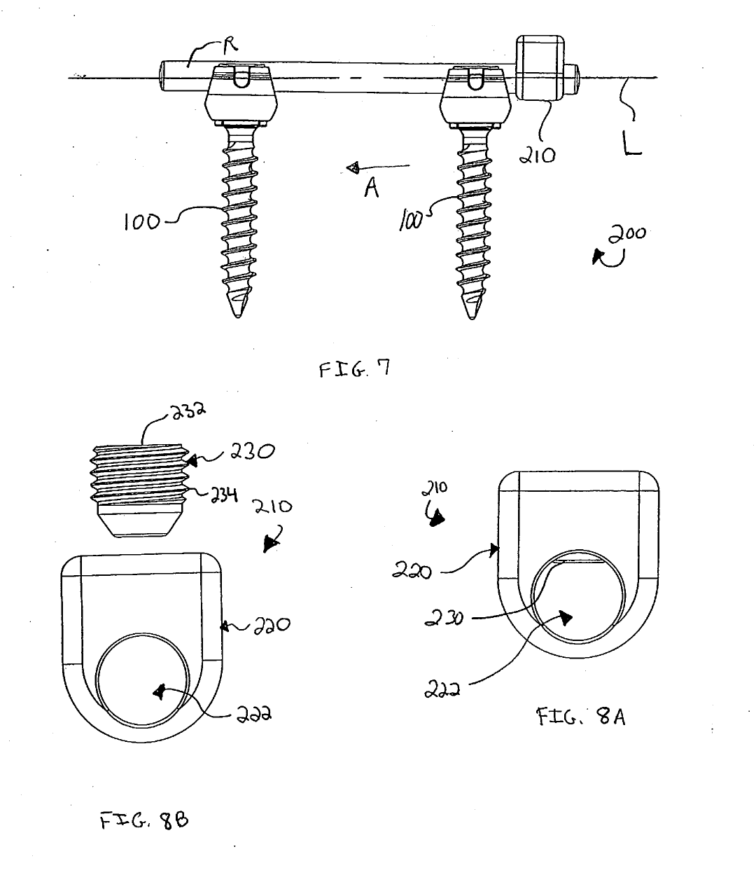

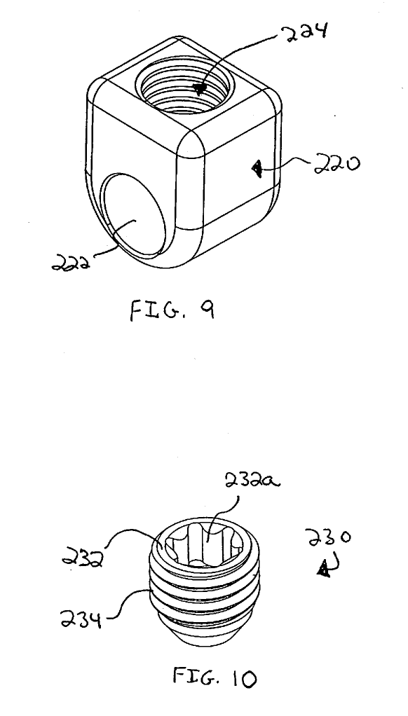

Referring now to FIGS. 4A and 4B, the pedicle screw 10 includes a shank 16 having a helical thread 14 formed thereon. A cutting portion 12 is formed at a distal end of the pedicle screw 10. A head 18 is located at a proximal end of the pedicle screw 10. The head 18 has an outer diameter that is greater than the outer diameter of the shank 16. On the top surface of the head 18, a recess 20 is formed. The recess 20 is illustrated with a six-pointed star configuration for receiving the operative end of a suitable driving tool, but it is contemplated that other configurations may be used. A neck 16a extends between a bottom surface of the head 18 and the beginning of the helical thread 14. As configured, the neck 16a is unthreaded. As shown, at least a portion of the diameter of the neck 16a is less than the diameter of the bottom of the head 18 and the major diameter of the threaded portion of the shank 16.

Referring again to FIGS. 1A-1C, the spinal fixation device 100 will now be discussed as assembled for use. The collet 70 is seated atop the head 18 of pedicle screw 10. The opening at the bottom of collet 70 is dimensioned and configured for receiving the head 18. As such, the collet 70 and the head 18 are rotatable and pivotable in relation to each other, thereby allowing the pedicle screw 10 to be repositioned in a plurality of orientations relative to the collet 70. The combination of the collet 70 and pedicle screw 10 is inserted into the coupling 50. The pin 30 aligns the collet 70 and the coupling 50 for maintaining a fixed relationship between them (FIGS. 1C and 5). As assembled, the pedicle screw 10 is rotatable and pivotable in relation to the collet 70 and the coupling 50.

Referring now to FIG. 6, additional features of the assembled spinal fixation device 100 will be discussed. The coupling 50 includes an inner annular lip 55 that is beveled. The lip 55 extends upwards and inwards from a bottom outer edge of the coupling 50. Additionally, the collet 70 includes an annular beveled lip 79 that also extends upwards and inwards from bottom outer edge of the collet 70. As shown in FIG. 6, angle α measures the angle of the beveled lip 79 from centerline C to the beveled lip 79. Angle α may measure between 25 and 65 degrees. In an embodiment, angle α is approximately equal to 45 degrees. Angle β measures the angle of the beveled lip 55 from the centerline C to the beveled lip 55. Angle β may measure between 32 and 72 degrees. In an embodiment, angle β is approximately equal to 52 degrees. By providing the coupling 50 and the collet 70 with beveled lips 55, 79, there is a reduced interaction between the head 18 and the coupling 50 and/or the collet 70. In addition, the pedicle screw 10 has a neck 16a with a length and diameter that cooperate with the beveled lips 55, 79 for reducing interaction therebetween. That is, the length of the non-threaded neck portion 16a of the pedicle screw 10 extends a distance from the bottom of the head 18 to a point beyond the beveled lip 79 of the of the collet 70 and beveled lip 55 of the coupling 50, which together with the selected diameter of the neck 16a permits maximum angular motion of the pedicle screw 10 relative to the collet 70 and coupling 50. This creates a smooth transition zone between the unthreaded neck 16a and the collet 70 and the coupling 50. By reducing the interference between the neck 16a and the beveled lips 55, 79 in combination with the reduced interaction between the head 18 and the beveled lips 55, 79, the pedicle screw 10 defines a cone of at least 70° with respect to a centerline “C” of the spinal fixation device (FIG. 6). In another embodiment, the pedicle screw 10 has a conical range of motion that is at least 90°. In a further embodiment, the pedicle screw 10 has a conical range of motion that is at least 95°.

Specifically, the pedicle screw 10 is capable of being repositioned from a first position (FIG. 6) throughout a plurality of angular positions with respect to the centerline “C”. The angular displacement with respect to the centerline “C” is shown as angleθ. Angle θ is at least 70°. In other embodiments, angle θ is in a range between about 80° and about 95°. As such, the pedicle screw 10 moves relative to the centerline “C” (i.e. off axis) in a range of about 35° to about 47.5°.

Referring now to FIGS. 7-10, one embodiment of a spinal fixation assembly is generally designated as 200. The spinal fixation assembly 200 includes a spinal rod “R”, one or more spinal fixation devices 100, and a locking element 210. The spinal rod “R” may be positioned transverse to the screw 10 upon engagement with the saddle 78 of the collet 70 of each spinal fixation device 100.

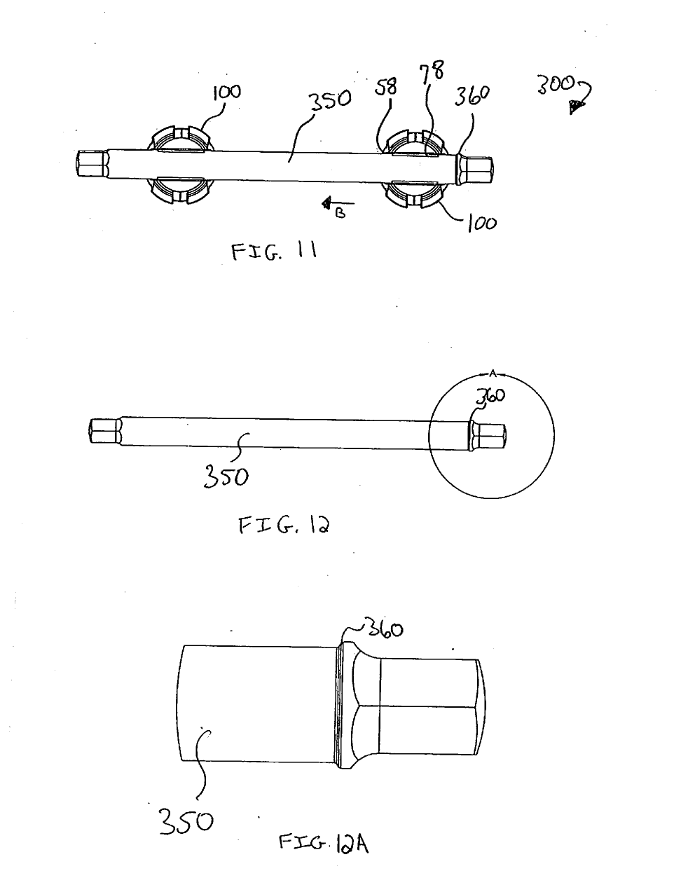

The locking element 210 is mounted to the spinal rod “R” and is configured to prevent axial translation of the spinal rod “R” along the longitudinal axis “L” thereof. The width of the locking element 210 is greater than the width of the saddle 78 and/or saddle 58 such that when the locking element 210 abuts saddle 78 and/or saddle 58, the locking element 210 prevents the spinal rod “R” from axially translating in one or both directions. Where one locking element 210 is positioned on one end of the spinal rod “R”, the spinal rod “R” is prevented from moving in one of the axial directions such as the direction illustrated by arrow “A” in FIG. 7. In embodiments, a plurality of locking elements 210 may be positioned on the spinal rod “R” on opposing sides of one or more spinal fixation devices 100 such that the spinal rod “R” is prevented from moving in both axial directions. The locking element 210 includes a body 220 defining a passage 222 adapted to receive the spinal rod “R” therethrough in locking engagement therewith. The locking element 210 includes a locking screw 230 for selectively locking the locking element 210 to the spinal rod “R.” The locking screw 230 rotates into locking engagement with the spinal rod “R.” The locking screw 230 includes a head 232 and a shaft 234. As best shown in FIG. 10, the head 232 defines a recess 232a. The recess 232a is illustrated with a six-pointed star configuration for receiving the operative end of a suitable driving tool (not shown), but it is contemplated that other configurations may be used. As best shown in FIG. 9, the locking screw 230 is disposed in threaded engagement with a threaded bore 224 defined within the body 220 of the locking element 210. In particular, the shaft 234 of the locking screw 230 includes threads for threadably engaging the threaded bore 224 of the body 220.

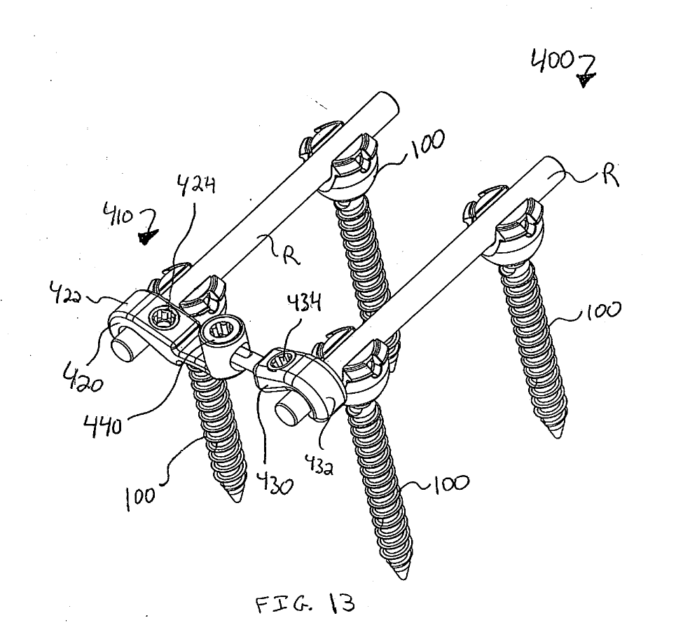

With reference to FIGS. 11-12A, one embodiment of a spinal fixation assembly is generally designated as 300. The spinal fixation assembly 300 includes one or more spinal fixation devices 100 and a spinal rod 350. The spinal rod 350 has one or more locking elements 360 configured to prevent axial translation of the spinal rod 350 when the spinal rod 350 is positioned within saddle 78 and/or saddle 58 of the spinal fixation device 100. The locking element 360 prevents the spinal rod 350 from axially translating in one direction. In this embodiment, the spinal rod 350 is prevented from moving in the axial direction illustrated by arrow “B” in FIG. 11. In embodiments, the spinal rod 350 may include a plurality of locking elements 360 positioned on opposing sides of the one or more spinal fixation devices 100 such that the spinal rod 350 is prevented from moving in both axial directions. The one or more locking elements 360 may be an annular ring disposed around the spinal rod 350 (FIG. 12A). The diameter of the one or more locking elements 360 is greater than the width of saddle 78 and/or saddle 58 such that when the one or more locking elements 360 abut saddle 78 and/or saddle 58, the one or more locking elements 360 prevent the spinal rod 350 from axially translating.

Referring now to FIG. 13, one embodiment of a spinal fixation assembly 400 includes two or more spinal fixation devices 100, two or more spinal rods “R”, and one or more locking elements 410. Locking element 410 includes first and second rod grasping members 420, 430 interconnected by a locking screw 440. Each rod grasping member 420, 430 is adapted to removably attach to the spinal rods “R” by grasping arms 422, 432 and lock thereto by locking screws 424, 434 for preventing each spinal rod “R” from axially translating. One example of such a locking element 410 is disclosed in commonly owned U.S. patent application Ser. No. 12/125,612, the content of which is hereby incorporated by reference herein.

It will be understood that various modifications may be made to the embodiments of the presently disclosed device. While the present description relates primarily to taper lock screws, it will be understood that the principles of the disclosure also apply to other types of screws, including set screw rod locking mechanisms. Therefore, the above description should not be construed as limiting, but merely as exemplifications of embodiments. Those skilled in the art will envision other modifications within the scope and spirit of the present disclosure.

What is claimed is:

1. A spinal fixation assembly, comprising:
a spinal rod defining a longitudinal axis;
at least one spinal fixation device, comprising:
a coupling having an opening extending therethrough;
a screw mounted to the coupling and positionable within a first vertebral body; and
a collet receivable in the opening of the coupling and defining a saddle for engaging the spinal rod;
and
a locking element mounted to the spinal rod and configured to prevent axial translation of the spinal rod in at least one direction along the longitudinal axis thereof.

2. The spinal fixation assembly of claim 1, wherein the spinal rod is positioned transverse to the screw upon engagement with the saddle of the collet.

3. The spinal fixation assembly of claim 1, further comprising a second spinal fixation device having a collet defining a saddle wherein the spinal rod is positionable within the saddles of each spinal fixation device.

4. The spinal fixation assembly of claim 1, wherein the locking element includes a body defining a passage adapted to receive the spinal rod therethrough in locking engagement therewith.

5. The spinal fixation assembly of claim 4, wherein the locking element includes a locking screw for selectively locking the locking element to the spinal rod.

6. The spinal fixation assembly of claim 5, wherein the locking screw rotates into locking engagement with the spinal rod.

7. The spinal fixation assembly of claim 5, wherein the locking screw is disposed in threaded engagement with a threaded bore defined within the body of the locking element.

8. The spinal fixation assembly of claim 1, wherein the width of the locking element is greater than the width of the saddle such that when the locking element abuts the saddle, the locking element prevents the spinal rod from axially translating in at least one direction.

9. A spinal fixation assembly, comprising:
at least one spinal fixation device, comprising:
a coupling having an opening extending therethrough;
a screw mounted to the coupling and positionable within a first vertebral body; and
a collet receivable in the opening of the coupling and defining a saddle; and
a spinal rod having at least one locking element configured to prevent axial translation of the spinal rod in at least one axial direction when the spinal rod is positioned within the saddle of the collet of the at least one spinal fixation device.

10. The spinal fixation assembly of claim 9, wherein the at least one locking element is an annular ring around the spinal rod.

11. The spinal fixation assembly of claim 9, wherein the diameter of the at least one locking element is greater than the width of the saddle such that when the at least one locking element abuts the saddle, the at least one locking element prevents the spinal rod from axially translating in at least one axial direction.

12. A spinal fixation assembly, comprising:
a spinal rod defining a longitudinal axis;
at least one spinal fixation device, comprising:
a coupling having an opening extending therethrough, the coupling configured to engage the spinal rod;
a screw mounted to the coupling and positionable within a first vertebral body; and
a locking element mounted to the spinal rod and configured to prevent axial translation of the spinal rod in at least one direction along the longitudinal axis thereof.

13. The spinal fixation assembly of claim 1, wherein the locking element includes a body defining a passage adapted to receive the spinal rod therethrough in locking engagement therewith.

14. The spinal fixation assembly of claim 13, wherein the locking element includes a locking screw for selectively locking the locking element to the spinal rod.

15. The spinal fixation assembly of claim 14, wherein the locking screw rotates into locking engagement with the spinal rod.

16. The spinal fixation assembly of claim 15, wherein the locking screw is disposed in threaded engagement with a threaded bore defined within the body of the locking element.
